(19) United States
(12) Patent Application Publication
(10) Pub. No.: US 2017/0122795 A1
(43) Pub. Date: May 4, 2017

(54) Mobile Health and Weight Management System

(71) Applicant: Leigh Stephanie Hernan, Scituate, MA (US)

(72) Inventor: Leigh Stephanie Hernan, Scituate, MA (US)

(21) Appl. No.: 14/930,130

(22) Filed: Nov. 2, 2015

(51) Int. Cl. G01G 19/44; G01S 19/42; G01G 19/12; A61B 5/0402; A61B 5/00; A61B 5/02; B60Q 9/00; B60N 2/44

(52) U.S. Cl. CPC G01G 19/44; B60Q 9/00; G01S 19/42; B60N 2/44; A61B 5/0402; A61B 5/486; A61B 5/742; A61B 5/02; G01G 19/12

(57) ABSTRACT

A weight management system for use by vehicle operators. A set of seats measuring devices to measure the weight of a user, or a set of user weights. The system then suggests exercises and diets to assist in weight management. Additionally the system can take a variety of pulmonary readings through the steering wheel and can send alerts if dangerous levels are found. The system can also be used to display various advertisements for fitness and dietary companies. The system is also designed to monitor when a user enters an unhealthy food venue and alert the user of numerous healthier choices within a designated radius.

### CROSS-REFERENCE TO RELATED APPLICATION

This application takes priority from and claims the benefit of U.S. Provisional Patent Application Ser. No. 62/073,434 filed on Oct. 31, 2014, the contents of which are herein incorporated by reference.

### BACKGROUND OF THE INVENTION

Field of the Invention

The present invention relates generally to health management devices and more particularly to an onboard personal health management system for vehicles.

Description of the Related Art

Obesity and other related health problems are growing concerns in the United States and occur of obesity have increased well over 50% in the last 40 years. The sedentary lifestyle of the majority of the workforce is a major contributing factor to the epidemic. This is especially true of those individuals who operate vehicles for a living, or who regularly commute for long distances behind the wheel of a vehicle.

Concurrently, numerous weight management systems exist on the market but they often require excessive amounts of devoted time to achieve results. The disclosed system seeks to avoid this limitation by providing an in-vehicle health management system. By providing an in vehicle health management system a user's health can be monitored and managed effectively without interfering with the users daily activities. The objective is achieved through integration of a variety of health management devices into the user's vehicle.

Although concurrent systems may include a scale built into the seat of a car for the purpose of weighing the driver, there are clear differences between these inventions and the system disclosed herein systems. Concurrent systems fail to encompass or disclose a seat scale which may be programmed to keep track of weight, offer dietary and exercise suggestions and incorporate advertisements from weight controllers.

Some systems discloses an invention that basically tracks a user's weight and warns the user if they are in danger of being obese. These systems sometimes offer the option of contacting healthcare providers to help the scale user battle the overweight problem. Concurrent systems may also offers the user potential routines and exercise activities to help the user battle the overweight problem. One aspect of the instant system that greatly differs from concurrent systems includes a mechanism wherein the instant system may integrate programs such as Weight Watchers™ and may allow for weight programs to advertise how they can help the user. Thus, the disclosed system affords the user more options to choose the best dieting plan.

Another aspect of the instant system that greatly differs from concurrent systems is the ability for the steering wheel to track heart rate and record other pulmonary readings in order to track history and stress of the body. With such an attribute, the automobile program will not only track the weight, but also the vitals of the user and in conjunction offer suggestions to a healthier user.

### SUMMARY OF THE INVENTION

The instant apparatus and system, as illustrated herein, is clearly not anticipated, rendered obvious, or even present in any of the concurrently disclosed mechanisms, either alone or in any combination thereof. A versatile system, method and series of apparatuses for creating and utilizing a health management and weight control system.

It is an additional object of the instant invention to provide a multifaceted, dynamic health management system.

It is also an object of the instant invention to provide a multifaceted, dynamic health management system for use in vehicles.

It is a further object of the instant invention to provide for a weight measurement device integrated in a vehicle operator's seat.

It is a further object of the instant invention to provide for a health data measuring device integrated into the steering wheel of a vehicle.

It is also an object of the instant invention to record the input user health data.

It is a further object of the instant invention to report progress of user health by analyzing user input data over time and providing feedback to the user.

It is a further object of the instant invention to provide health management suggestions to the user so that the user may improve their individual heath and tendencies.

It is a further object of the instant invention to provide health management suggestions to the user based on geographical location determine through use of an integrated GPS system.

It is an additional object of the instant invention to provide a method of use of an in vehicle health management system.

It is a further object of the instant invention to introduce a system that comprises a set of scales programmed to record the weight of a respective user and concurrently provide various systems linking the user with dietary and exercise suggestions and incorporating advertisements from weight controllers as well as offering integration to programs such as Weight Watchers® to also allow for weight programs to advertise the assistance that may be provided to the user.

It is also an object of the instant invention to readily incorporate the ability to track heart rate, vital signs and monitor other pulmonary readings in order to track history and stress of the body.

It is an additional object of the instant invention to readily incorporate the ability for the a series of monitoring and sensing apparatuses and transducers located within a vehicle steering wheel to track heart rate.

It is also an object of the instant invention wherein the system is disposed to detect when a vehicle enters an unhealthy food venue, as rated by an accredited rating organization for nutritional values of foods and food preparation. The system will further alert the operator of numerous healthier choices within a designated radius

There has thus been outlined, rather broadly, the more important features of the versatile mobile health and weight management system embodiments in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty, which characterize the invention, are pointed out with particularity in the claims annexed to and forming a part of this disclosure. For a better understanding of the invention, its operating advantages, and the specific objects attained by its uses, reference should be made to the accompanying drawings and descriptive matter in which there are illustrated preferred embodiments of the invention.

The above summary of the present invention is not intended to describe each disclosed embodiment or every implementation of the present invention. The Figures, Detailed Description and Examples which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 illustrates a flow diagram of the operation of the Health Management System.

FIG. 2 illustrates a flow diagram of the data transmission of the Health Management System.

FIG. 3 illustrates a flow diagram of the menu initialization of the Health Management System incorporated with the touchscreen.

FIG. 4 illustrates a flow diagram of the menu flow for an existing user.

FIG. 5 illustrates a flow diagram of the menu flow for a new user.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE SEVERAL EMBODIMENTS

For the following defined terms, these definitions shall be applied, unless a different definition is given in the claims or elsewhere in this specification. All numeric values are herein assumed to be modified by the term “about”, whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings wherein like reference numerals indicate like elements throughout the several views. The drawings, which are not necessarily to scale, depict illustrative embodiments of the claimed invention.

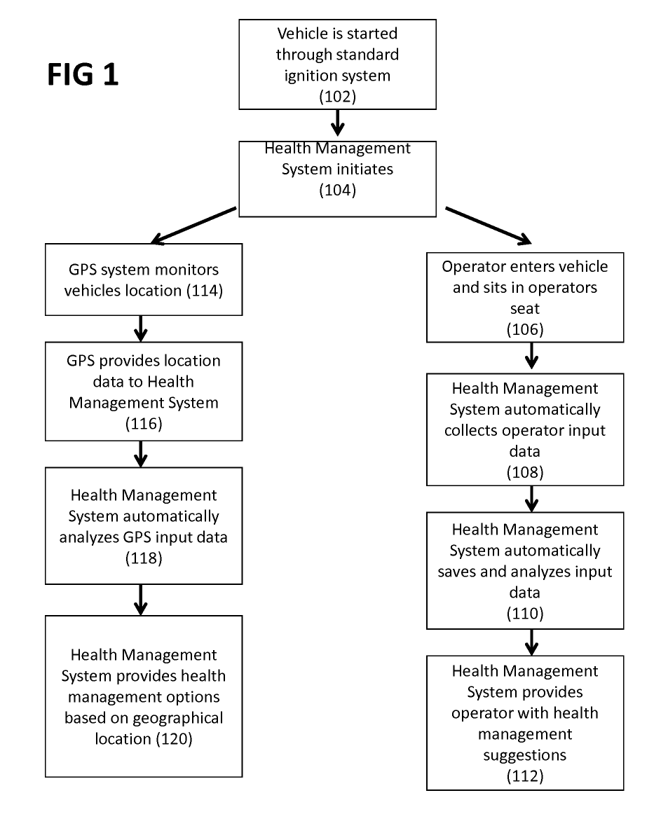

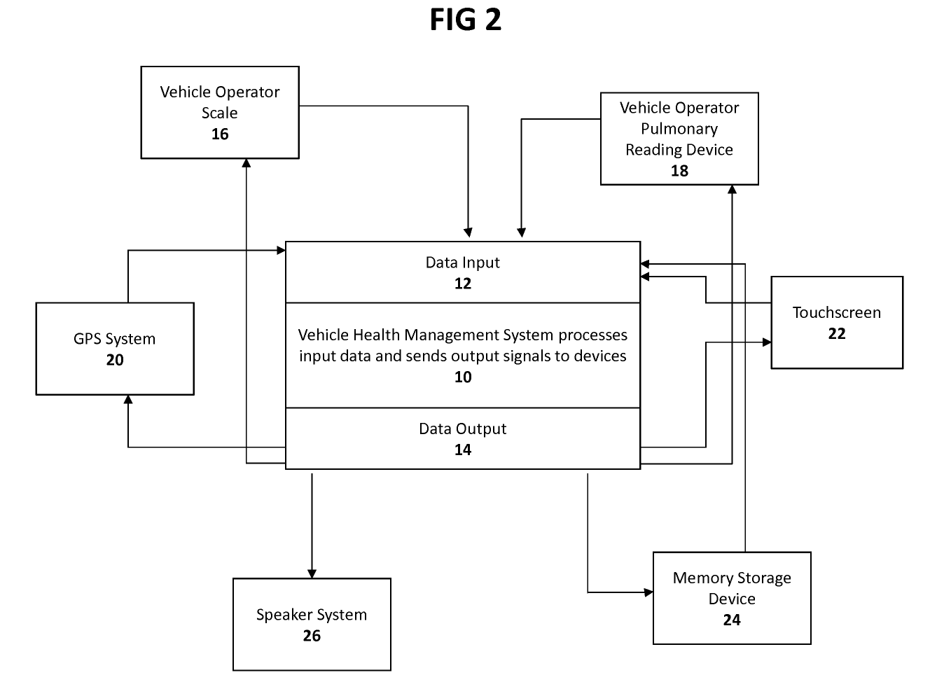

FIG. 1 illustrates a flow diagram of the operation of one embodiment of the Health Management System and FIG. 2 illustrates a flow diagram of the data transfer within the health management system 10. At step 102 a vehicle with the integrated Health Management System 10 is started through the standard ignition procedure. At ignition of the vehicle the Health Management System 10 begins an initiation procedure at step 104. The vehicle operator then sits in the driver's seat in step 106. When the initiation procedure of step 104 has concluded the Health Management System 10 begins the process of collecting the driver's input data 12 at step 108.

Further, Driver data input 12 is collected through various inputs parameters. Driver data inputs 12 may include, but are not limited to, a scale 15 incorporated in the driver's seat and a set of electrocardiography readings through vehicle operator pulmonary reading devices 18 mounted in the steering wheel. At step 110, the driver input data 12 is saved and analyzed by the Health Management System 10. At step 110, the collected driver input data 12 is compared to previously collected data as well as various health standards. At step 112, the Health Management System 10 provides feedback and health management suggestions to the driver.

A set of Feedback includes, but is not limited to, changes in driver health input data 12 and potential health concerns. The health management system 10 also provides suggestions to improve the driver's personal health. The suggestions include, but are not limited to, a set of exercise options, dietary suggestions, healthy restaurant choices and professional help suggestions.

After initiation of the Health Management System 10 at step 104, the system begins to monitor vehicle location data through use of an integrated GPS system 20 at step 114. At step 116, the GPS system 20 transmits the vehicle location input data 12 to the Health Management System 10. At step 118, the Health Management System 10 analyzes the GPS input data 12. At step 120, the Health Management System 10 provides health management options outputs to the driver. Health management options may include, but are not limited to, nearby and proximate in vehicle distance healthy eating locations, recreational activities and health professional locations.

In one embodiment when the system is activated, the program will provide health tip and preventative verbiage, and even utilize such systems as GPS in order to assist the user in keeping with proper fitness and eating habits. Specifically, if the system detects that a vehicle operator is on the verge of pulling up to what the system determines to be (and is programmed to sort) an unhealthy food venue, the system will utilize GPS to then alert the operator of numerous healthier choices within a designated radius. In one embodiment, the user may limit the system to a one mile radius of the proximal unhealthy food venue destination.

In another embodiment, the system may list the choices by way of numerous differing categories including, but not limited to, grocery stores, small choice restaurants, or full services restaurants. Additionally, the system may list the choices by way of differing categories including, but not limited to, food types such as salads, grilled foods, and yogurt. Moreover, the system may list the choices by way of pricing, as well as many other parameters an operator deems useful.

In an alternative, the instant system may access other existing systems and services to enhance the user capability. One genre of system for access could be consumer rating systems that rate food quality and preparation of different facilities. These could be used as a basis for recognition of what is healthier approach, thereby the choice is not interdependent upon only the instant system's suggestion, but also rather what studies have shown and recommended for a higher rating.

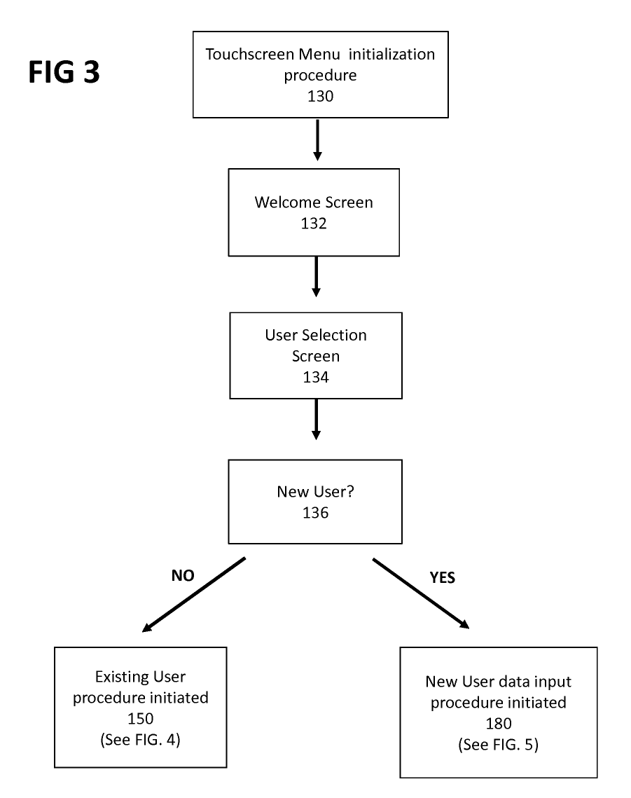

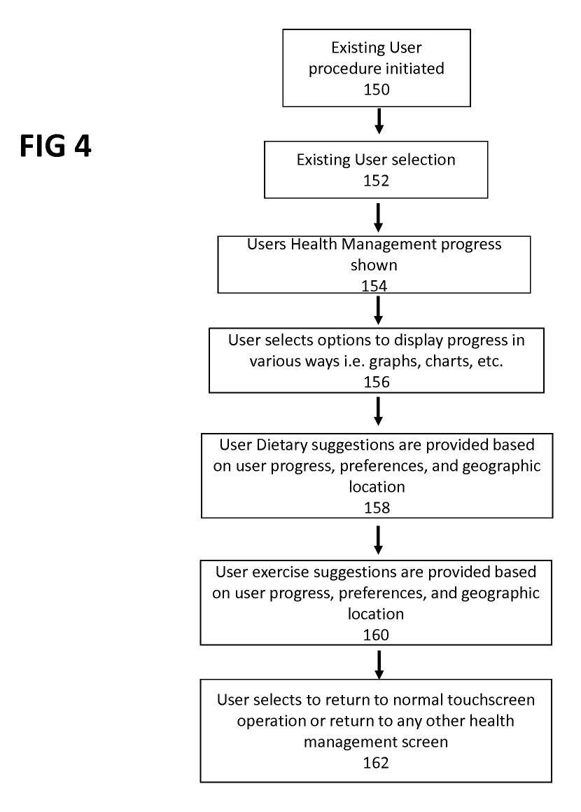

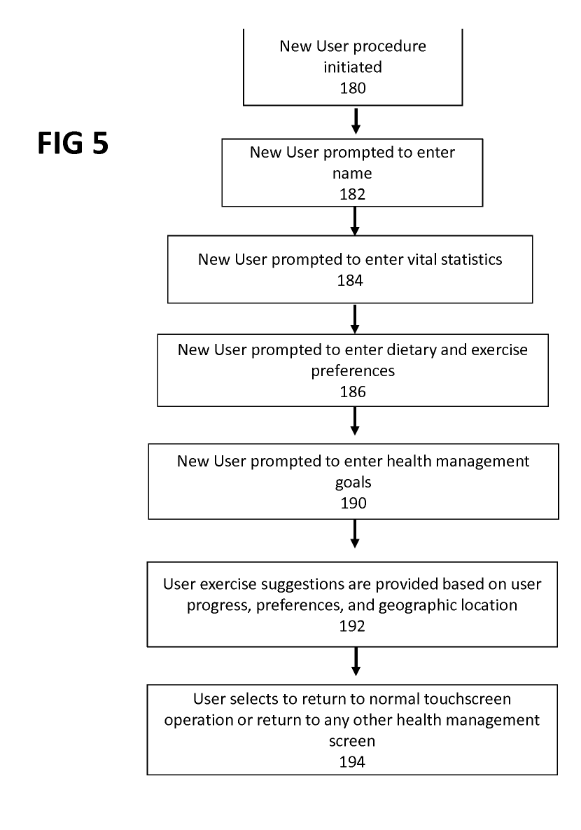

Furthermore, FIG. 3, FIG. 4, and FIG. 5 comprise flow diagrams that illustrate the progression of the Health Management Systems 10 menu located on the touchscreen 22. At step 130, the touchscreen 22 initializes at the start of the vehicle by the user. At step 132, the touchscreen 22 displays a welcome screen. The welcome screen displays a greeting to the user and acknowledges the Health Management System 10 has started. At step 134, the touchscreen 22 displays a user selection screen. At step 136, the user is shown a question “New User?” and asked to answer YES or NO. The user answer dictates the direction in which the menu will proceed.

FIG. 4. illustrates the flow from a NO selection. Selecting NO at step 136, the user indicates an existing user and the existing user procedure is initiated at step 150. At step 152, the user selects the corresponding existing user. At step 154, the user's health management progress is shown. This is a result of the Health Management System 10 automatically saving and analyzing the user input data.

At step 156, the user selects a variety of ways to display their health management progress. The various displays can be in the form of charts, graphs, medical assessments, and other ways of displaying health statistics. At step 158, the touchscreen 22 displays dietary suggestions to the user. The dietary suggestions are a result of the user's preferences, geographical location, and health statistics. At step 160, the touchscreen 22 displays exercise suggestions to the user. Thus, the exercise suggestions may result of from a combination of the user selected preferences, geographical location, and health statistics to apply varied sources. At step 162, the user can select to return to normal touchscreen 22 operation as programmed by the car manufacturer or return to previous health management system 10 displays.

FIG. 5 illustrates a flow chart of the new user input procedure 180. The new user input procedure 180 is initiated upon a selection of Yes at step 136. At step 182, the new user enters their name using the keyboard incorporated into the touchscreen 22. At step 184, the new user enters their vital statistics. The vital statistics include user age, height, weight, health conditions, and any other health data that is valuable to the health assessment. At step 186, the new user is prompted to enter their dietary tendencies and exercise tendencies.

This information allows the system to provide health management suggestions that are tailored to the particular user's habits. At step 190, the user is prompted to enter their health management goals. The health management goals allow a program to be created for the user to achieve their desired goals. At step 192, the health management system 10 provides exercise suggestions based on the user's entered preferences and geographic location. At step 194, the user is provided the ability to either select to return to normal touchscreen 22 operation or return to any other health management screen display.

Moreover, the vehicle may be equipped with on board exercise equipment such as tension systems for arms and legs. Some systems may include, but are not limited to, leg adductor systems, leg abductor systems and hand gripping systems. Neck tensioning systems may also be utilized.

In conclusion, herein is presented a weight management system, preferably for use in a variety of vehicles. The invention is illustrated by example in the drawing figures, and throughout the written description. It should be understood that numerous variations are possible, while adhering to the inventive concept. Such variations are contemplated as being a part of the present invention.

What is claimed is:

1. An integrated vehicle health management system comprising:
a set of weight measurement devices mounted in at least one vehicle seat;
a set of pulmonary reading devices;
a set of output display apparatuses;
a set of input devices;
an on-board vehicle microprocessor apparatus;
a set of random access memory and a display mechanism connected to the microprocessor;
a set of storage devices;
a global positioning system;
at least one memory storage device;
wherein the set of storage devices receive and retain the set of data from the set of weight measurement device and the set of data from the set of pulmonary reading devices; and wherein the microprocessor accesses sets of data from the set of weight measurement device and the set of data from the set of pulmonary reading devices and the microprocessor the data to provide a set of health indications to a user; and,
wherein the system is disposed to detect when a vehicle enters an unhealthy food venue, via an electronic communication with the global positioning system and an accredited rating organization for nutritional values of foods and food preparation, and alert the operator of numerous healthier choices within a designated radius.

2. The integrated vehicle health management system of claim 1, wherein the set of weight measurement devices mounted in at least one vehicle seat activates through a standard vehicle ignition procedure.

3. The integrated vehicle health management system of claim 1, wherein the set of pulmonary reading devices are mounted in a vehicle steering wheel.

4. The integrated vehicle health management system of claim 1, wherein the global positioning system transmits a vehicle location.

5. The integrated vehicle health management system of claim 1, further comprising electrical connections for connecting the set of weight measurement device, the set of pulmonary reading devices, the set of input devices to the on-board vehicle microprocessor apparatus.

6. The integrated vehicle health management system of claim 1, further comprising electrical connections for connecting the on-board microprocessor apparatus to the set of random access memory and display mechanism.

7. The integrated vehicle health management system of claim 1, wherein the set of input devices comprises data input from a vehicle's user.

8. The integrated vehicle health management system of claim 7, wherein the data input from the vehicle's user selected from the group consisting of a set of electrocardiography readings, a set of weight readings, and a set of pulmonary readings.

9. The integrated vehicle health management system of claim 1, wherein the set of health indications provided by the microprocessor comprises a set of health management feedback.

10. The integrated vehicle health management system of claim 9, wherein the set of health management feedback comprises exercise options, dietary suggestions, healthy restaurant choices, nutrition information, and professional help suggestions.

11. The integrated vehicle health management system of claim 1, wherein the global positioning system locates health management options for the vehicle's user.

12. The integrated vehicle health management system of claim 11, wherein the health management options comprises, proximate in vehicle distance, healthy eating locations, recreational activities, and health professional locations.

13. A method for utilizing the integrated vehicle health management system of claim 1, comprising the steps of:
activating the integrated vehicle health management system through a standard vehicle ignition procedure;
applying at least one user's weight to at least one vehicle seat;
beginning a vehicle initiation procedure;
collecting user's input data;
saving the user's input data in the memory storage device;
connecting the user's input data to the on-board vehicle microprocessor apparatus
analyzing the user's input data;
comparing the user's input data with various health standards;
providing the user with health management feedback;
locating health management options with the global positioning system.
